(19) United States
(12) Patent Application Publication
(10) Pub. No.: US 2009/0281477 A1
(43) Pub. Date: Nov. 12, 2009

(54) ELECTROPORATION DEVICE AND METHOD

(75) Inventors: PAUL W. MIKUS, RANCHO SANTA MARGARITA, CA (US); WILLIAM C. HAMILTON, JR., QUEENSBURY, NY (US); WILLIAM M. APPLING, GRANVILLE, NY (US)

Correspondence Address: ANGIODYNAMICS, INC., 603 QUEENSBURY AVENUE, QUEENSBURY, NY, 12804, US

(73) Assignee: ANGIODYNAMICS, INC., QUEENSBURY, NY (US)

(21) Appl. No.: 12/437,843

(22) Filed: May 8, 2009

Related U.S. Application Data
(60) Provisional application No. 61/051,832, filed on May 9, 2008.

(51) Int. Cl. A61N 1/30; A61B 18/14

(52) U.S. Cl. 604/21; 606/41

(57) ABSTRACT

Disclosed herein are methods and devices configured for pulsed voltage ablation that have two or more voltage delivery regions separated along a probe by electrically insulating regions. The electrically insulating regions are of sufficient lengths to allow the voltage delivery regions to be oppositely energized for delivering voltage pulses to treat cells within a predetermined ablation volume that can be delineated through representations indicating pulsed voltage ablation thresholds for cells.

### RELATED APPLICATIONS

This application claims priority under 35 U.S.C. 119(e) to U.S. Provisional Application No. 61/051,832, filed May 9, 2008, which is incorporated herein by reference.

### BACKGROUND OF THE INVENTION

1. Field of the Invention

The present application relates generally to devices and methods for tissue treatment using Irreversible Electroporation (IRE). More specifically, the application relates to devices and methods for treatment of tissue through the application of pulsed electric fields that create nonthermal cellular effects and that can be applied at a level of sufficient strength so as to result in ablation of tissue.

2. Description of the Related Art

The application of an electric field to transiently permeabilize cells is a method known as reversible electroporation. In such as a case, membrane defects are created and later reseal, allowing a time when macromolecules can be introduced across the cell membrane. This has been used, for example, to insert genes into cells (electrogenetherapy), and to insert anti-cancer drugs into cells (electrochemotherapy). A primary goal of reversible electroporation is to lead to cellular defects that allow passage of macromolecules awhile still allowing cell survival.

Irreversible electroporation (IRE) is a novel method of applying electrical fields across tissue through a delivery of pulses that effectively result in membrane permeabilization and in cell necrosis. IRE has been discussed in the following publication, which is hereby incorporated by reference: Rubinsky B., Onik G., Mikus P. “Irreversible Electroporation: A New Ablation Modality—Clinical Implications.” Technology in Cancer Research and Treatment. Vol. 6(1):37-48 (2007).

### BRIEF SUMMARY OF THE INVENTION

Devices and methods of the present application can be suitable for deliver of gradients of electric fields in a pulsed manner for certain tissue treatments, including but not limited to ablation purposes. For pulsed electric field ablation treatments, a selected ablation voltage threshold with a field strength of 0.25 kV/cm or greater can outline an ablation volume within the electric field gradient, which emanates radially from the voltage delivery regions with decreasing field strengths. During pulsed electric field ablation treatments, tissue at a specific point within the ablation volume can be subjected to an electric field having a field strength equal to or greater than a selected ablation voltage threshold, with tissue points closer to the single probe of the device being subjected to voltages fields of greater strengths.

One embodiment of the device includes a first voltage deliverer member wherein the first voltage delivery member includes a single tissue piercing tip and a first voltage delivery region: a second voltage delivery member extending along the probe, wherein the second voltage delivery member includes a second voltage delivery region; and an electrically insulating region extending along the probe and that separates the first and second voltage delivery regions. The first and second voltage delivery regions and the electrically insulating region in one embodiment have substantially equivalent outer diameters and are configured for electroporative ablation of tissue a tissue volume. In another embodiment the ablation involves a mammalian tissue volume. In yet another embodiment the ablation volume can have a ratio of diameter to length being 1:2 or greater.

One example method of tissue treatment includes: utilizing the device disclosed herein, positioning the first and second voltage delivery regions within or adjacent to a selected volume of tissue, and energizing the device to deliver pulsed electric fields to the selected volume of tissue.

An additional method of tissue treatment includes: utilizing a device with a probe having two or more coaxially arranged voltage delivery regions, positioning the two or more voltage delivery regions within or adjacent to a selected volume of tissue, and energizing the device to deliver pulsed electric fields to the selected volume of tissue. The voltage pulses in certain embodiments can be delivered to generate an electric field strength of 0.25 kV/cm with a pulse duration of up to 100 microseconds, and with a time between pulses being 0.15 seconds or longer. In other embodiments the voltage pulses can be delivered to generate an electric field strength of up to 2.5 kV/cm. In yet other embodiments the electric field strength can be between 2.5-3.0 kV/cm. The pulses can be delivered in a nonthermal method. In one embodiment the pulses are delivered so as to ensure that the temperature of the tissue did not exceed 50° C.

### BRIEF DESCRIPTION OF THE SEVERAL VIEWS OF THE DRAWINGS

A more complete understanding of the present invention can be derived by referring to the detailed description when considered in connection with the following illustrative figures. In the figures, like reference numbers refer to like elements or acts throughout the figures. Throughout the specification, the term “distal” is consistently used in reference to the device or portion of the device farthest from the user and “proximal” refers to the end closest to the user of the device.

FIG. 1 is a plan view of an electroporation device designed for tissue treatment having a first voltage delivery region and a second voltage delivery region separated by an electrically insulating region.

FIG. 2 is an exploded plan view of the device in FIG. 1 showing spacing and insulating as well as voltage delivery components of the probe.

FIG. 3A is an enlarged partial longitudinal sectional view of A-A from FIG. 1 showing a distal portion of the probe.

FIGS. 3B-3D are cross sectional views of the probe of FIG. 3A, taken along section lines B-B, C-C, and D-D therein, respectively.

FIG. 4 is a plan view of an electroporation device designed for tissue treatment having a first, a second, and a third voltage delivery region each separated by an electrically insulating region.

FIGS. 5-8 are plan views of pulsed electric field gradients in the form of Finite Element Analysis (FEA) representations showing voltage delivery regions of tissue treatment devices of the present application, under certain design parameters and operating conditions as detailed hereinafter. In each of FIGS. 5, 6, 7, and 8, two voltage delivery regions are shown as example embodiments.

FIGS. 9-10 are plan views of pulsed electric field gradients in the form of Finite Element Analysis (FEA) representations showing voltage delivery regions of tissue treatment devices of the present application, under certain design parameters and operating conditions as detailed hereinafter. In each of FIGS. 9 and 10. three voltage delivery regions are shown as example embodiments.

FIG. 11 is a general perspective view of the device of FIG. 1.

FIG. 12 is a partial plan view of a proximal portion of the device of FIG. 1.

FIG. 13 is an enlarged view of the distal portion of the device of FIG. 1 showing a blunt tip for a bipolar device.

FIG. 14 is an enlarged view of the distal portion of a monopolar device having a blunt tip.

Elements and acts in the figures are illustrated for simplicity and have not necessarily been rendered according to any particular sequence or embodiment.

### DETAILED DESCRIPTION OF ACHE INVENTION

In the following description, and for the purposes of explanation, numerous specific details are set forth in order to provide a thorough understanding of the various aspects of the invention. It will be understood, however, by those skilled in the relevant arts, that the present invention can be practiced without these specific details. In other instances, known structures and devices are shown or discussed more generally in order to avoid obscuring the invention. In many cases, a description of the operation is sufficient to enable one to implement the various forms of the invention. It should be noted that there are many different and alternative configurations, devices and technologies to which the disclosed inventions can be applied. The full scope of the inventions is not limited to the examples that are described below.

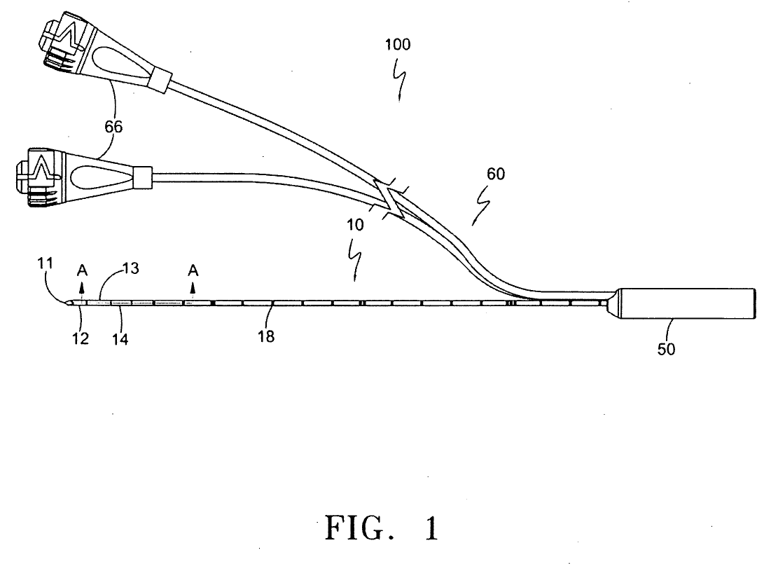

FIG. 1 shows a device 100 designed for tissue treatment (including but not limited to pulsed electric field ablation and electroporative ablation), as a non-limiting example incorporating features herein described. Device 100 as shown contains a holding member such as a handle 50 and an elongated member such as a probe 10 that terminates within handle 50. Probe 10 as shown has a body portion extending from a tissue piercing tip 11 into handle 50. Device 100 has bundled cables 60 exiting from handle 50 in a distal direction that is coupled to a power supply (such as a high voltage pulse generator, not shown in FIG. 1), for example, via connectors 66.

Probe 10 can include at least two voltage delivery regions 12 and 14, which can be electrically insulated from each other and disposed along the length of the body portion of probe 10. Probe 10 can further include at least one electrically insulating region 13 that separates the at least two voltage delivery regions 12 and 14 in a manner sufficient (such as having a length sufficient) to prevent electrical shorting as well as to prevent arcing between voltage delivery regions 12 and 14. Electrically insulating region 13 can have a diameter substantially the same as or smaller or larger than those of voltage delivery regions 12 and 14. Other features along the body portion of probe 10 can include indexing methods 18, such as depth markings.

The plurality of voltage delivery regions can be independently or inter-dependently configured to be energized with a predetermined polarity, as long as at least two of the voltage delivery regions are configured to be energized with opposite polarities. It has been observed that when voltage delivery regions 12 is configured to be positively (cathodically) energized while voltage delivery region 14 is configured to be negatively (anodically) energized, electrical arcing at tip 11 occurred less than if the polarity is reversed (i.e., with region 12 being negative (anodic) while region 14 being positive (cathodic).

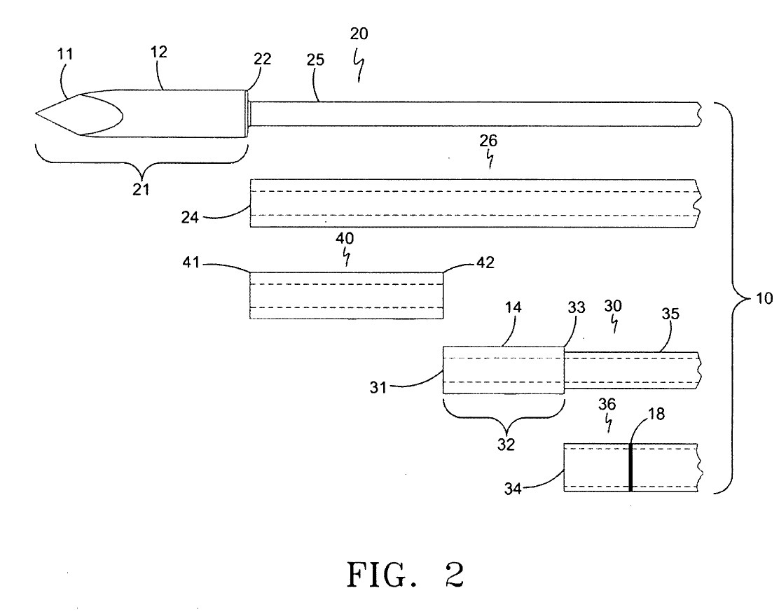

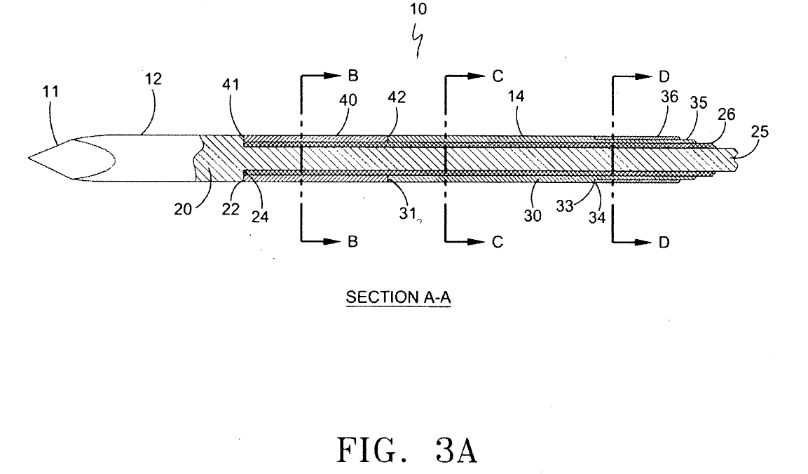

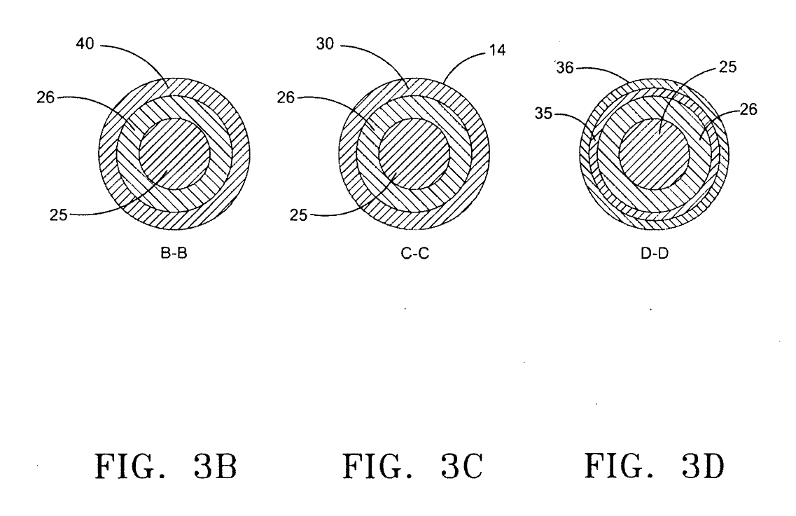

FIGS. 2, 3A, 3B, 3C, and 3D all depict a distal portion of probe 10, with FIGS. 2 and 3 showing a longitudinal view and FIGS. 3B, 3C, and 3D showing a transverse view of the respective portion outlined in FIG. 3A (B-B, C-C, and D-D for FIGS. 3B, 3C, and 3D respectively). FIGS. 3B-3D show the orientation of electrically conducting and insulating portions that can be arranged in certain embodiments so as to provide the probe with a substantially similar diameter throughout its length. This probe can include a first voltage delivery member 20, a first electrically insulating member 26, an optional spacing member 40, a second voltage delivery member 30, and a second electrically insulating member 36. Two or more of these components can be coaxially arranged with respect to each other. Voltage delivery member 20 in one embodiment can have a solid construction, without any lumen or opening. Alternatively, in additional embodiments, voltage delivery member 20 can have one or more longitudinal lumens as well as openings at its distal end or on its sides that are in communication with the longitudinal lumen or lumens. Tip 11 can have a beveled profile to enable or facilitate percutaneous application of probe 10.

Voltage delivery member 20 can include a distal portion 21 for voltage delivery that includes voltage delivery region 12 and tip 11 (which can be part of voltage delivery region 12), and a proximal portion 25 that can be electrically conducting for electrically coupling voltage delivery region 12 to a power supply (not shown). Portion 21 can extend to edge 22, having a uniform diameter along a majority of its length (excluding tip 11). The uniform diameter can be substantially the same as the outermost diameters shown in FIGS. 3B-3D, so that probe 10 can be substantially uniform in diameter along its length. Portion 25, extending from portion 21 into handle 50, can be substantially smaller in diameter (such as by 0.02 inches or greater) than portion 21 (excluding tip 11). The diameter of portion 25 can be 0.02 inches or greater, or 0.03 inches or greater. The diameter of portion 25 can be 90% of that of portion 21 (excluding tip 11) or less, 80% or less, 70% or less, 60% or less, or 50% or less. Portions 21 and 25 can have the same or different compositions, and can independently be comprised of one or more electrically conductive materials, including one or more metals and alloys thereof, such as various grades of stainless steel. Voltage delivery member 20 can have one or more lumens there through and one or more openings (such as at the distal end, on the side of portion 21) for delivery of substances (including but not limited to at least one of infusion media, solutions or suspensions containing one or more therapeutic as well as diagnostic agents, hydrogels, colloidal suspensions containing nanoparticles as well as microparticles). In certain embodiments the substances are delivered to increase the conductivity of the tissue and in others is delivered to increase the efficiency of ablation. In other embodiments the substances are released to alter the conductivity of tissue. In other embodiments the device is capable of extracting a substances selected from the group consisting of tissue, fluids, medium, solutions, suspensions, therapeutics, hydrogels, nanoparticles, and microparticles.

Electrically insulating member 26 can be coaxially disposed about portion 25 of voltage delivery member 20. Electrically insulating member 26 can be coextensive distally with portion 25, and extend from edge 24 into handle 50. Electrically insulating member 26 can include one or more layers of the same or different electrically non-conductive materials. Electrically insulating member 26 can electrically insulate portion 25 to prevent electrical shorting as well as to prevent arcing thereof, which can adversely affect treatment efficiency as well as efficacy. Use of multiple layers as well as coatings to form electrically insulating member 26 reduces or eliminates the occurrence of pin holes and damage occurring during the manufacturing process. When assembling probe 10, electrically insulating member 26 can be applied onto portion 25, among other methods, by sliding on and shrink-wrapping one or more tubular structures (such as sleeves as well as tubing) of thermoplastics, as well as by forming one or more surface coatings (such as vapor deposition, spraying, dipping, as well as molding). Suitable electrically non-conductive materials can have a dielectric strength of 10 MV/m or greater, such as 15 MV/m or greater, or 20 MV/m or greater. Electrically non-conductive materials for electrically insulating member 26 include polyethylene terephthalate, polyimides, polyamides, polyamide-imides, singly and in combinations of two or more. Electrically insulating member 26 can have a uniform outer diameter. Thickness of electrically insulating member 26 can be 0.05 inches or less. In alternative embodiments, member 26 is 0.03 inches or less in diameter.

As indicated, FIG. 3A shows a longitudinal view of the distal portion of the probe of an embodiment of the device and FIGS. 3B-3D show the orientation of electrically conducting and insulating portions that can be arranged in certain embodiments so as to provide the probe with a substantially similar diameter throughout its length. Starting with FIG. 3A and FIG. 3B for example, the optional spacing member 40 can be coaxially disposed about electrically insulating member 26. Spacing member 40 can be placed adjacent to portion 21 of voltage delivery member 20. Edge 41 of spacing member 40 can be positioned adjacent to edge 22 of voltage delivery member 20. Spacing member 40 can include one or more layers of the same or different electrically non-conductive materials. Spacing member 40 can be used to space voltage delivery region 12 from other voltage delivery regions along probe 10. Spacing member 40 can be used to achieve a uniform diameter along probe 10. It has been observed that probe 10 with spacing member 40 is less prone to electrical shorting as well as less prone to arcing than probe 10 without spacing member 40. When assembling probe 10, spacing member 40 can be applied onto electrically insulating member 26, among other methods, by sliding or molding on one or more tubular structures (including sleeves as well as tubing) of thermoplastics. Suitable electrically non-conductive materials for spacing member 40 include medical grade thermoplastics that are sufficiently rigid for deployment and retraction through tissue as well as sufficiently heat-resistant. Suitable electrically non-conductive materials can have a dielectric strength of 10 MV/m or greater, such as 15 MV/m or greater, or 20 MV/m or greater. Electrically non-conductive materials for spacing member 40 include thermosets and thermoplastics, such as polyether ether ketone, polyphenylene sulfide, fluoropolymers, and polyamide-imides. Spacing member 40 can be a cylinder, have an outermost diameter that is substantially the same as the outermost diameters shown in FIGS. 3B-3D, so that probe 10 can be substantially uniform in diameter along its length.

Electrically insulating member 26 and spacing member 40 in combination physically separates and electrically insulates voltage delivery member 20 from other voltage delivery members (including voltage delivery member 30 as described herein) of probe 10. In certain examples, electrically insulating member 26 (or a layer thereof) and spacing member 40 (or a layer thereof), comprised of the same or different electrically non-conductive materials, can be fabricated as a single-piece tubular structure rather than separate pieces to simplify the assembly of probe 10. The single-piece, electrically insulating member can have a distal cylindrical portion that is greater in outer diameter and wall thickness than a proximal cylindrical portion. A central lumen passing through the distal and proximal portions of the single-piece electrically insulating member can have a substantially uniform diameter that is equal to or greater than the outer diameter of portion 25 of voltage delivery member 20. Non-limiting methods of making a single electrically insulating piece that includes electrically insulating member 26 and spacing member 40 include extrusion (including co-extrusion), molding (including co-injection molding), and others known to one skilled in the art. In other examples, the optional spacing member 40 can be omitted from probe 10.

As shown in FIG. 3A and FIG. 3C, Voltage delivery member 30 can be a tubular structure coaxially disposed about electrically insulating member 26, having an inner diameter equal to or greater than the outer diameter of electrically insulating member 26. Voltage delivery member 30 can be placed adjacent to the optional spacing member 40. Edge 31 of voltage delivery member 30 can be positioned adjacent to edge 42 of the optional spacing member 40. Voltage delivery member 30 can include a distal portion 32 for voltage delivery that includes voltage delivery region 14, and a proximal portion 35 that can be electrically conducting for electrically coupling voltage delivery region 14 to a power supply (not shown). Portion 32 can extend from non-piercing edge 31 to edge 33, having a uniform outer diameter along its length. The uniform outer diameter can be substantially the same as the outermost diameters shown in FIGS. 3B-3D, so that the body portion of probe 10 can be substantially uniform in diameter along its length. Portion 35, extending from portion 32 into handle 50, can be smaller in diameter (in certain embodiments by 0.02 inches or less and in other embodiments by 0.01 inches or less) than portion 32. In additional embodiments the diameter of portion 35 can be 0.04 inches or greater, and in other embodiments 0.05 inches or greater. In certain embodiments the diameter of portion 35 can be 95% of that of portion 32 or less, and in other embodiments be 90% or less. Portions 32 and 35 can have the same or different compositions, and can independently be comprised of one or more electrically conductive materials, including one or more metals and alloys thereof, such as various grades of stainless steel. Voltage delivery member 30 can have one or more lumens there through and one or more openings (including at the distal end as well as on the side of portion 32) for delivery of substances (including but not limited to infusion media, solutions or suspensions containing one or more therapeutic as well as diagnostic agents, hydrogels, colloidal suspensions containing nanoparticles as well as microparticles). In certain embodiments the substances are delivered to increase the conductivity of the tissue and in others is delivered to increase the efficiency of ablation. In other embodiments the substances are released to alter the conductivity of tissue.

Electrically insulating member 36 can be coaxially disposed about portion 35 of voltage delivery member 30. Electrically insulating member 36 can be coextensive distally with portion 35, and extend from edge 34 into handle 50. Electrically insulating member 36 can include one or more layers of the same or different electrically non-conductive materials. Electrically insulating member 36 can electrically insulate portion 35 to prevent electrical shorting as well as to prevent arcing thereof, which can adversely affect treatment efficiency as well as efficacy. Use of multiple layers as well as coatings to form electrically insulating member 36 reduces or eliminates the occurrence of pin holes and damages therein during the manufacturing process. When assembling probe 10, electrically insulating member 36 can be applied onto-portion 35, among other methods, by sliding on and shrink-wrapping one or more tubular structures (including sleeves as well as tubing) of thermoplastics, as well as by forming one or more surface coatings (including but not limited to vapor deposition, spraying, dipping, as well as molding). Suitable electrically non-conductive materials can have a dielectric strength of 10 MV/m or greater, such as 15 MV/m or greater, or 20 MV/m or greater. Electrically non-conductive materials for electrically insulating member 36 include polyethylene terephthalate, polyimides, polyamides, polyamide-imides, and combinations of two or more thereof. Electrically insulating member 36 can have a uniform outer diameter (as depicted in FIG. 3D) that is substantially the same as those shown in FIGS. 3B-3C, so that the body portion of probe 10 can be substantially uniform along its length. Thickness of electrically insulating member 36 can in certain embodiments be 0.05 inches or less, and in additional embodiments can be 0.03 inches or less. Electrically insulating member 36 can include a plurality of indexing methods 18 (including depth markings) that are detectable (including visible) to the operator.

Optionally, one or more of voltage delivery regions 12 and 14 can be rendered more echogenic than other regions (including the electrically insulating region 13, outer surface of electrically insulating member 36) along probe 10. Certain embodiments include non-limiting methods for echogenicity enhancement including particle blasting, echogenic coating. perforating, chemical etching, and laser etching. In certain embodiments, microabrasive blasting is applied to voltage delivery regions 12 and 14 to achieve a depth of 70 microns.

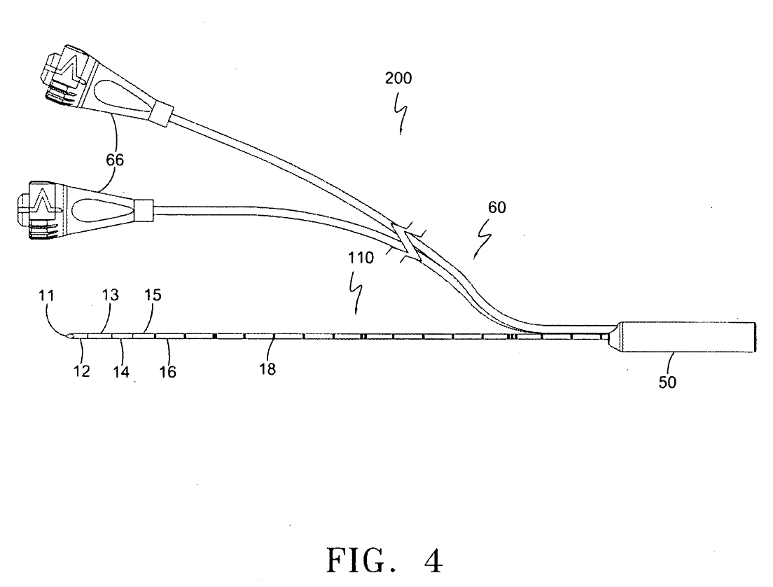

FIG. 4 shows an additional embodiment, as device 200, designed for tissue treatment (including but not limited to pulsed electric field ablation and electroporative ablation), as a non-limiting example incorporating features herein described. Device 200 is depicted with a handle 50 and a probe 110 that terminates proximally within handle 50. Probe 110 is shown to have, in a distal portion thereof, a tissue piercing tip 11. Device 200 can have cables 60 exiting from handle 50 in a distal direction that is coupled to a power supply (including a voltage pulse generator, not shown), for example, via connectors 66. Probe 110 can include, along its length, at least three voltage delivery regions 12, 14 and 16. The voltage deliver) regions 12, 14 and 16 can be separated and electrically insulated from each other by at least two electrically insulating regions 13 and 15. Other features along probe 110 can include indexing methods 18 such as depth markings.

Each of voltage delivery regions 12, 14 and 16 can be independently or inter-dependently configured to be energized with a predetermined polarity, as long as at least two of the voltage delivery regions are configured to be energized with opposite polarities. In certain embodiments each of voltage delivery regions 12, 14 and 16 are electrically coupled to one of three cables and one of three separate connectors, to be independently energized as well as polarized. In additional embodiments, voltage delivery region 12 is oppositely energized with respect to one or both of voltage delivery regions 14 and 16. In other embodiments, voltage delivery region 14 is oppositely energized with respect to one or both of voltage delivered regions 12 and 16. In other embodiments, voltage delivery region 16 can be oppositely energized with respect to one or both of voltage delivery regions 12 and 14. In additional embodiments, only two of voltage delivery regions 12, 14 and 16 are oppositely energized at any given time, while the others are not energized. In certain embodiments, voltage delivery region 12 is cathodically energized, and in other embodiments region 12 is not energized. Certain embodiments include at least one of the following pattern of charge (polarization) for voltage delivery regions 12, 14 an 16: (+, −, −), (+, −, +), (+, +, −), (−, +, +), (−, +, −), (−, −, +), (+, −, X), (+, X, −), (−, +, X), (−, X, +), (X, +, −) and (X, −, +) where X represents no polarization. Any one of such patterns can be chosen exclusively throughout a procedure. Alternatively, a combination of two or more of these patterns can be chosen in a predetermined series, randomly, or manually for any one or more portions of a procedure. In various embodiments of FIG. 1 or 4, voltage delivery regions can be independently polarized as well as independently energized so as to ensure that a circuit is formed for current movement from any of the voltage delivery regions to any of the voltage delivery regions. In certain embodiments a cathodic polarization in the voltage delivery region including the tip (at the distal end of the probe) will be energized such that current flows from the tip to a voltage delivery region independently charged anodically that is not at the tip and is closer to the proximal half of the probe (closes to the distal portion of the handle) than to the tip. In certain embodiments where multiple probes are utilized together, independently energizing and independently polarizing can be used to ensure current flows from any voltage delivery region on one probe to any voltage delivery region on another probe, at any point along the length of the voltage delivery member containing a voltage delivery region.

The tissue treatment devices illustrated as well as described herein are configured for delivery of pulsed electric field gradients to tissue surrounding the two or more voltage delivery regions. The pulsed electric field gradients can emanate away from the energized voltage delivery regions as substantially uniformly gradients of decreasing field strengths. Shapes and sizes of pulsed electric field gradients can depend in part on specific combinations of the following: 1) amplitude of the supplied voltage pulses, 2) dimensions (including lengths as well as diameters) of each of the voltage deliver regions, and 3) dimensions (including lengths as well as diameters) of each of the electrically insulating regions separating the voltage delivery regions. Shapes and sizes of the pulsed electric field gradients can be identified using non-limiting algorithms and software such as Finite Element Analysis (FEA) (COMSOL® Version 3.3, Comsol, Inc., Burlington, Mass.).

Pulsed voltage ablation thresholds, electric field strengths to which cells in a target tissue are exposed substantially damage, destroy, render dead or otherwise metabolically inactivate the cells, can be identified for any tissue type. For example, a pulsed voltage ablation threshold of 0.25 kV/cm has been demonstrated for destruction of certain cancer cells in vitro. This has been discussed in the following publication, which is hereby incorporated by reference: Miller L., Leor J., Rubinsky B. “Cancer Cell[ ] Ablation with Irreversible Electroporation.” Technology in Cancer Research and Treatment, Vol. 4(6), 699-705, 2005. As such, pulsed voltage ablation volumes of any given configuration of voltage delivery regions can be identified, for example, as the FEA-calculated electric field gradient outlined by a predetermined pulsed voltage ablation threshold (in kV/cm).

In certain embodiments, configurations of the voltage deliverer regions and the electrically insulating regions can: increase or maximize a ratio of diameter to length of tie pulsed voltage ablation volume (with the pattern being wide and short, or being or approaching substantially spherical), and can minimize the occurrence of electrical arcing between different voltage deliver), regions. This can have broad clinical applications. In certain embodiments the ratio of diameter to length of the pulsed voltage ablation volume can be 1:2 or greater, and in other embodiments can be 4:7 or greater, and in other embodiments can be 3:5 or greater. In other examples, this ratio of the pulsed voltage ablation volume can be 1:4 or greater, and in other embodiments can have a ratio that is at least one of: 2:7 or greater, 1:3 or greater, as well as 3:7 or greater.

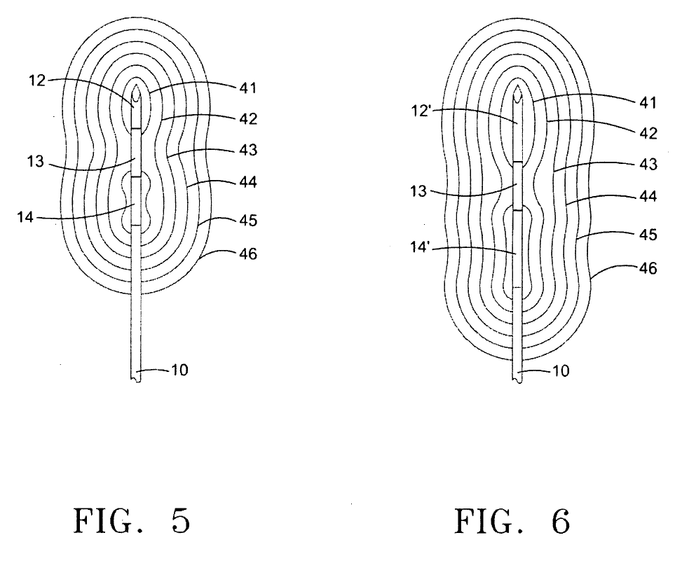

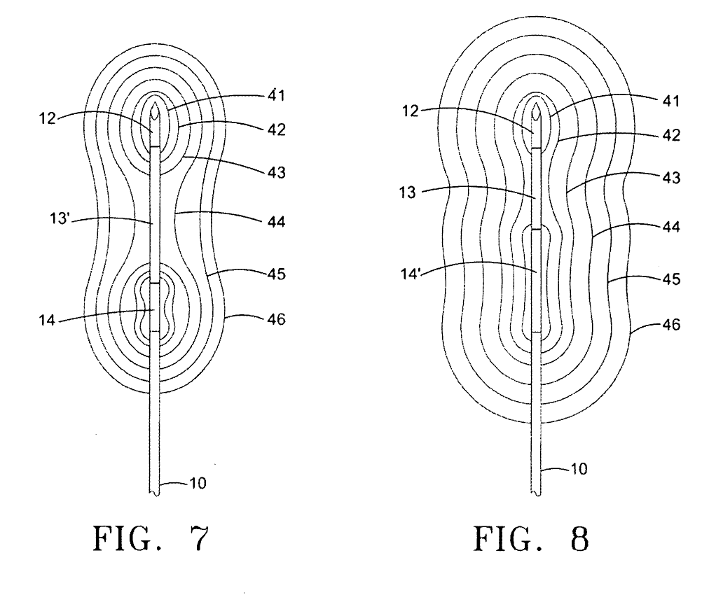

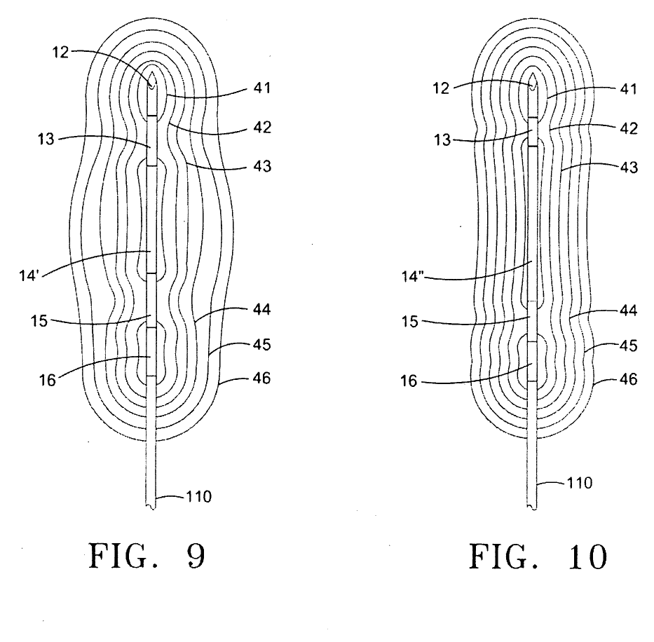

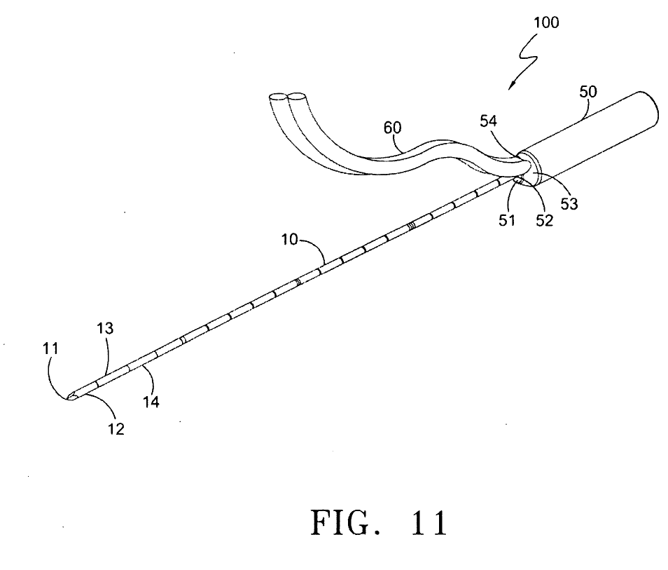

FIG. 5-10 are orthogonal views of pulsed electric field gradients in the form of Finite Element Analysis (FEA) representations showing voltage delivery regions of tissue treatment devices of the present application, under certain design parameters and operating conditions as detailed hereinafter. In each of FIGS. 5, 6, 7 and 8, two voltage delivery regions are shown as example embodiments, and in FIGS. 9 and 10 three voltage delivery regions are shown as example embodiments. The FEA representations show different patterns occurring when there are variations in the number of voltage delivery regions (here 2 shown in FIGS. 5-8 and three in FIGS. 9 and 10, though additional voltage delivery regions are conceived) or there are differences in lengths or ratios between the voltage deliver regions and the electrically insulating regions. The specifics of each FEA diagram as shown in FIGS. 5-10 are described in detail in the text of the following paragraphs below. FIG. 5 shows a pattern where an electrically insulating region 13 is approximately the same length as each of two voltage delivery regions (the distal voltage delivery region 12 and the proximal voltage delivery region 14). FIG. 6, shows a pattern where an insulating region 13 is much shorter than (approximately half as long as) the length as each of two voltage delivery regions (12′ and 14′). FIG. 7, shows a pattern where an electrically insulating region 13′ is much longer than (approximately three times as long as) each of the two voltage delivery regions (12 and 14). FIG. 8 shows a pattern where one of the voltage delivery regions (proximal region 14′) is much longer than (approximately two times as long as) the insulating region 13 and much longer than (approximately two times as long as) the distal voltage delivery region 12, FIG. 9 shows a pattern resulting from the presence of three voltage delivery regions, where the most distal voltage delivery region 12 and the most proximal voltage delivery region 16) (and the two intervening electrically insulating regions) are each much shorter than the intervening voltage delivery region 14′ (that is approximately twice as long as each of 12, 13, 15, and 16) and where the order from proximal to distal of parts is 16,15,14′,13, 12, and FIG. 10 shows a pattern resulting from the presence of three voltage delivery regions, where intervening voltage delivery region 14″ is much longer than (approximately five times as long as) each of the additional voltage delivery regions (12 and 16) as well as the insulating regions (13 and 15), and where the order from proximal to distal of parts is 16,15,14″,13,12. More specific descriptions of each of FIGS. 5-10 follow in the paragraphs below.

FIGS. 5-10 show FEA representations of pulsed electric field gradients around voltage delivery regions. Specifically, FIGS. 5-8 illustrate various FEA representations that can be used to estimate pulsed voltage ablation volumes and that are: 1) outlined by a pulsed voltage ablation threshold of 0.25 kV/cm, 2) estimated by FEA using a 16-gauge (having a diameter of 0.065 inches) probe 10 with different configurations of the voltage delivery regions and the electrically insulating region, and 3) provided by a pulsed voltage of 2.7 kV. FIGS. 9-10 illustrate various FEA representations that can be used to estimate pulsed voltage ablation volumes and that are: 1) outlined by a pulsed voltage ablation threshold of 0.25 kV/cm, 2) estimated by FEA u using a 16-gauge probe 110 with different configurations of the voltage delivery regions and the electrically insulating regions, and 3) provided by a provided voltage of 2.7 kV. Other suitable gauge sizes that in certain embodiments include 14-22.

FIG. 5 is a FEA representation of a pulsed electric field gradient around voltage delivery regions. Specifically, electrically insulating region 13 has a length (of approximately 8 mm) substantially the same as or slightly greater than that of voltage delivery region 12 (approximately 7.5 mm) and voltage delivery region 14 (approximately 7 mm). When voltage delivery regions 12 and 14 are electrically coupled to a voltage pulse source (such as a voltage pulse generator) and are oppositely charged, they are capable of providing a pulsed electric field gradient depicted through isometric electric field strength lines 41, 42, 43, 44, 45, and 46 correspond to electric field strengths of 2.5 kV/cm, 1 kV/cm, 0.425 kV/cm, 0.25 kV/cm, 0.15 kV/cm, and 0.075 kV/cm, respectively. One skilled in the art would understand that the electric field radiates continuously from the voltage delivery regions of probe 10 outward with decreasing field strength, and includes the illustrated isometric electric field strength lines. In certain embodiments of ablation, there is a ratio describing the ablation volume, where the ratio of the diameter to length ablated would be 4:7 (such as a diameter of 2 cm and a length of 3.5 cm).

FIG. 6 is another FEA representation of a pulsed electric field gradient around voltage delivery regions. Specifically, electrically insulating region 13 has a length (approximately 8 mm) substantially less than (half as long as) that of each of the voltage delivery regions 12′ and 14′ (approximately 14 mm). When voltage delivery regions 12′ and 14′ are electrically coupled to a voltage pulse source (such as a voltage pulse generator) and are charged oppositely with a voltage difference of 3 kV, they are capable of providing a pulsed electric field gradient where the ablation volume achievable can extend from the probe out to line 46 in the pattern of line 46. Isometric electric field strength lines 41, 42, 43, 44, 45, and 46 correspond to electric field strengths of 2.5 kV/cm, 1 kV/cm, 0.425 kV/cm, 0.25 kV/cm, 0.15 kV/cm, and 0.075 kV/cm, respectively. One skilled in the art would understand that voltage gradients radiate continuously from the voltage delivery regions of probe 10 outward with decreasing field strength, and includes the illustrated isometric electric field strength lines. In certain embodiments of ablation, there is a ratio describing the ablation volume, where the ratio of the diameter to length ablated would equal 0.45.

FIG. 7 is a further FEA representation of a pulsed electric field gradient around voltage delivery regions. Specifically, electrically insulating region 13′ has a length (approximately 22 mm) substantially greater than (approximately 3 times as long as) that of voltage deliver) regions 12 and 14 (approximately 7 mm each). When voltage delivery regions 12 and 14 are electrically coupled to a voltage pulse source (such as a voltage pulse generator) and are charged oppositely with a voltage difference of 3 kV, they are capable of providing a pulsed electric field gradient where the ablation volume achievable can extend from the probe out to line 46 in the pattern of line 46. Isometric electric field strength lines 41, 42, 43, 44, 45, and 46 correspond to electric field strengths of 2.5 kV/cm, 1 kV/cm, 0.425 kV/cm, 0.25 kV/cm, 0.15 kV/cm, and 0.075 kV/cm, respectively. One skilled in the art would understand that voltage gradient radiates continuously from the voltage delivery regions of probe 10 outward with decreasing field strength, and includes the illustrated isometric electric field strength lines. In certain embodiments the ablation volume pattern will have a thinner central portion near electrically insulating region 13′, and can therefore resemble an ellipsoid cinched at the center or alternatively stated, can take on the shape of a dumbbell.

FIG. 8 is a further FEA representation of pulsed electric field gradient around voltage delivery regions. Voltage delivery region 12 has a length (approximately 7 mm) substantially equal to or slightly shorter than that of electrically insulating region 13 (approximately 8 mm) and substantially shorter than (half as long as) that of voltage delivery region 14′ (approximately 14 mm). When voltage delivery regions 12 and 14′ are electrically coupled to a voltage pulse source (such as a voltage pulse generator) and arc charged oppositely with a voltage difference of 3 kV, they are capable of providing a pulsed electric field gradient where the ablation volume achievable can extend from the probe out to line 46 in the pattern of line 46. Isometric electric field strength lines 41, 42, 43, 44, 45, and 46 correspond to electric field strengths of 2.5 kV/cm, 1 kV/cm, 0.425 kV/cm, 0.25 kV/cm, 0.15 kV/cm, and 0.075 kV/cm, respectively. One skilled in the art would understand that voltage gradients radiate continuously from the voltage delivery regions of probe 10 outward with decreasing field strength, and includes the illustrated isometric electric field strength lines.

FIG. 9 is a further FEA representation of a pulsed electric field gradient around voltage delivery regions. Three voltage delivery regions 12 (approximately 7 mm), 14′ (approximately 14 mm), and 16 (approximately 7 mm) are separated from each other by two electrically insulating regions 13 and 15 of substantially equal lengths (approximately 8 mm). When voltage delivery regions 12 and 16 are electrically coupled to a voltage pulse source (such as a voltage pulse generator) and are charged oppositely to voltage delivery region 14′, with a voltage difference of 3 kV, they are capable of providing a pulsed voltage gradient where the ablation volume achievable can extend from the probe out to line 46 in the pattern of line 46. Isometric electric field strength lines 41, 42, 43, 44, 45, and 46 correspond to electric field strengths of 2.5 kV/cm, 1 kV/cm, 0.425 kV/cm, 0.25 kV/cm, 0.15 kV/cm and 0.075 kV/cm, respectively. One skilled in the art would understand that voltage gradients radiate continuously from the voltage deliver, regions of probe 110 outward with decreasing field strength, and includes the illustrated isometric electric field strength lines. In certain embodiments the ablation volume can be seen with a bulging section at voltage delivery region 14′.

FIG. 10 is a further FEA representation of pulsed electric field gradient around voltage delivery regions. Three voltage delivery regions 12, 14″, and 16 having respective lengths of approximately 7 mm, 33 mm (4.7-fold of that of voltage delivery regions 12 and 16) and 7 mm are separated from each other by two electrically insulating regions 13 and 15 of substantially equal lengths (approximately 8 mm). When voltage delivery regions 12 and 16 are electrically coupled to a voltage pulse source (such as a voltage pulse generator) and are charged oppositely to voltage delivery region 14″, with a voltage difference of 3 kV, they are capable of providing a pulsed voltage gradient where the ablation volume achievable can extend from the probe out to line 46 in the pattern of line 46. Isometric electric field strength lines 41, 42, 43, 44, 45, and 46 correspond to electric field strengths of 2.5 kV/cm, 1 kV/cm, 0.425 kV/cm, 0.25 kV/cm, 0.15 kV/cm, and 0.075 kV/cm, respectively. One skilled in the art would understand that voltage gradient radiates continuously from the voltage delivery regions of probe 110 outward with decreasing field strength, and includes the illustrated isometric electric field strength lines.

The tissue treatment devices illustrated as well as described herein can be configured to have suitable as well as sufficient probe rigidity to reduce occurrence of kinks and breakages. In certain examples, probes 10 and 110 can have a gauge size of 22 or greater, while other embodiments include sizes 16 or greater. Certain embodiments have a probe or probes with a transverse area moment of inertia (MOI) of 3×10⁻⁷ in⁴ or greater, with other embodiments having probes with an MOI of 4×10⁻⁷ in⁴ or greater. Yet other embodiments have probes with a stress ratio of 9×10⁵ in³ or less, and certain embodiments have probes with a stress ratio of 7×10⁵ in³ or less. Additional embodiments have probes with a deflection factor of 9 lb·in² or greater, with certain embodiments having probes with a deflection factor of 13 lb·in² or greater.

The devices of the present application can include a single probe capable of containing two or more voltage delivery regions. The single-probe devices can be sufficient for certain pulsed voltage ablation procedures. Alternatively, the devices of the present application can include two or more probes constructed as illustrated as well as described herein, each carrying two or more voltage delivery regions. In other alternatives, multiple single-probe devices can be used in combination in other treatment procedures. When two or more probes are present in a device, or two or more probes of multiple devices are used in combination, the voltage delivery regions of different probes can in certain embodiments be aligned in parallel and in other embodiments will not be in parallel. Parallel probes can be configured to be sufficiently apart to reduce or eliminate occurrence of electrical shorting as well as arcing. Any two parallel voltage delivery regions of adjacent probes can be energized with the same polarity or opposite polarities. The combined electric field gradients resulting from energizing parallel probes can be calculated using FEA as described herein. Total treatment volumes (including total ablation volumes) can be identified within the combined electric field gradients, as described herein. In certain embodiments where multiple probes are utilized together, independently energizing and independently polarizing can be used to ensure current flows from any voltage delivery region on one probe to any voltage delivery region on another probe, at any point along the length of the voltage deliver, member containing a voltage delivery region.

The tissue treatment devices as illustrated as well as described herein can be suitable for treatment of conditions for various tissues, volumes, sizes and locations, including small to medium sized tissue volumes, and tissue volumes that are in close proximity to other non-targeted structures (that can include but are not limited to neuronal structures, vascular structures, duct structures, collagen-rich structures). Non-limiting examples of tissue masses to which the devices of the present application are applicable include benign tissue masses such as benign prostate hyperplasia (BPH) and uterine fibroids, as well as benign as well as malignant masses such as cancers and tumors of various tissue types (including but not limited to prostate, uterine, lung, liver, kidney, brain, head/neck, bone, stomach, colon, pancreas).

The tissue treatment devices as illustrated as well as described herein fixedly couple the two or more voltage delivery regions at predetermined distances from each other, reducing the demand on probe alignment and on the skill level of the operator, making the related procedures quick, easy, and widely adaptable, with reproducible and reliable outcomes (including the size and shape of the ablation volumes). The devices are configured such that the probe can be placed within or adjacent to the target tissue, enabling safe usage in situations where the tissue targeted for ablation is adjacent to critical as well as vital non-targeted structures (such as the urethra or neurovascular bundles). This feature can further broaden the applicability and adaptability of the related treatment procedures.

The tissue treatment devices as illustrated as well as described herein can enable safe delivery of pulsed voltage (including 1 kV or greater) treatment without endangering the patient or causing device malfunctions (such as shorting or arcing). The devices can further reduce tissue trauma and associated discomfort of the patient during the treatment procedures as compared to other devices using separate tissue-piercing needles containing each voltage delivery region. As such, duration and cost of subsequent recovery can be reduced following the treatment (including percutaneous) procedures. As described herein, the unique features as well as combinations thereof can reduce as well as eliminate device malfunctions such as electrical shorting as well as arcing, and can enhance patient safety as well as procedure efficiency and efficacy.

While the devices of the present application can be used for pulsed voltage ablation as described to engender irreversible electroporation, the can also be used to produce reversible electroporation (in certain embodiments to facilitate transportation of macromolecules across membranes), with appropriate modifications in operating parameters.

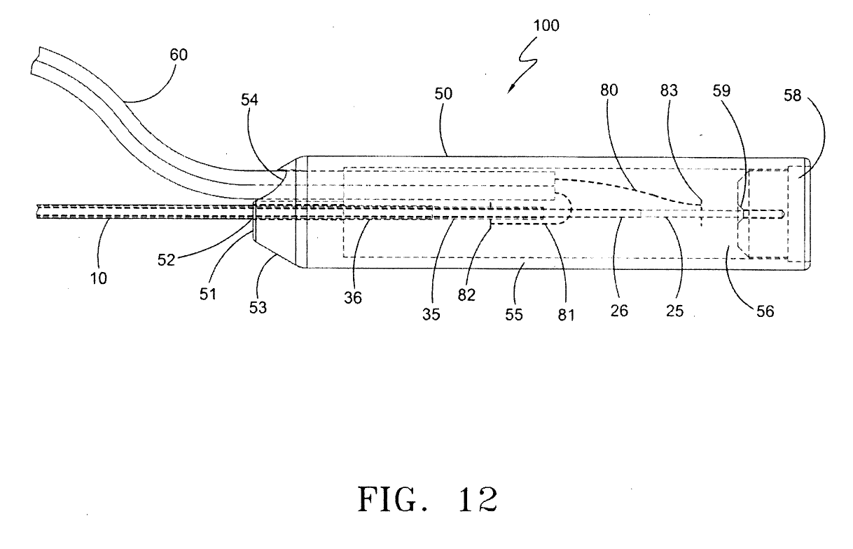

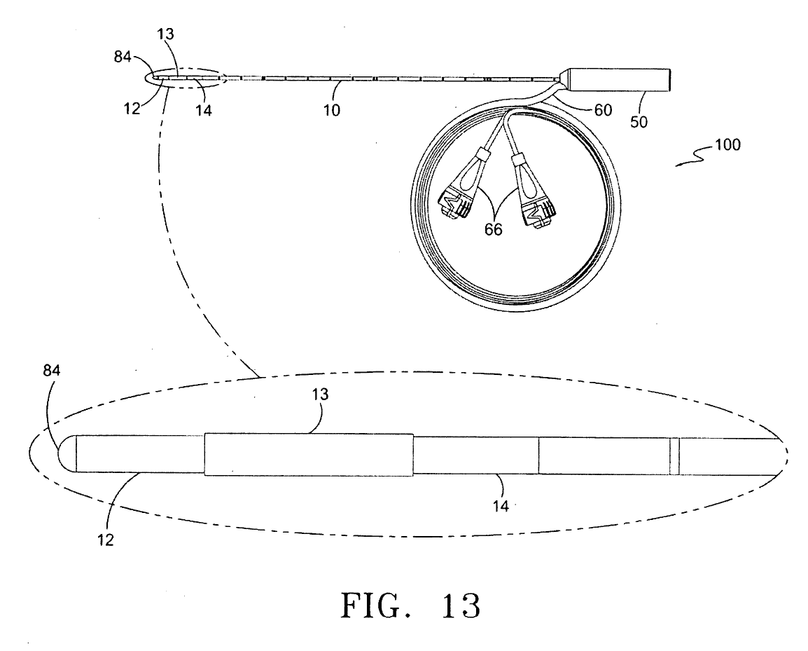

FIGS. 11 and 12 provide depictions of the exterior and interior components, respectively, of the device from FIG. 1. FIG. 11 is a general perspective view of the device of FIG. 1 and FIG. 12 is a perspective longitudinal partial view of a proximal portion of the device of FIG. 1.

More specifically, in referring to FIGS. 11-12, handle 50 can include a distal portion 53, a body portion 55, a proximal closing member 58 (such as a plug), and a cavity 56 defined by distal portion 53 and body portion 55. Distal portion 53, having a distal edge 51 thereof, can adopt a shape generally tapering distally. Distal portion 53 can have openings 52 and 54 positioned adjacent to each other and both in communication with cavity 56. Openings 52 and 54 can be facing substantially in a distal direction (toward tissue piercing tip 11) of device 100. Additional embodiments (not shown in FIGS. 11 or 12), can include three or more voltage delivery regions, an aspect that was depicted in device 200 from FIG. 4.

Opening 52, disposed on distal edge 51, can be configured for receiving probe 10, which can extend proximally into cavity 56 and terminate in a distal-facing recess 59 of plug 58. As such, at least opening 52 and plug 58 fixedly couple probe 10 with handle 50. Adhesives or other non-limiting bonding techniques can be used to render probe 10 immovable relative to handle 50. Additional embodiments (not shown in FIG. 11 or 12), can include three or more voltage delivery regions, an aspect that was depicted in device 200 (along probe 110) from FIG. 4.

Opening 54, optionally disposed on a tapering surface of distal portion 53, can be configured with various shapes known in the art including but not limited to elliptic or crescent shaped, and can be configured for receiving cables 60, which can extend proximally into cavity 56. One of the depicted cables 60 can be electrically coupled to proximal portion 25 (thus also to voltage deliver, region 12) through lead wire 80 and coupling methods 83, while another of cables 60 can be electrically coupled to proximal portion 35 (shown with electrically insulating member 36 shown disposed about portion 35) through lead wire 81 and coupling methods 82. Non-limiting examples of coupling methods 82 and 83 include soldering, lead wire wounding, electrically conductor lugs, and combinations thereof. The bonding joints are placed within body portion 55 during assembly. Note that electrically insulating region 13 and voltage delivery region 14 are shown for completeness in FIG. 11.

Cavity 56 can be filled with a flowable material (including but not limited to a liquid, semi-liquid, as well as a gel) and with a hardening material (in certain embodiments including at least one of a cross-linkable, polymerizable, or otherwise curable material) that is electrically insulating (such as epoxy) to secure and in certain embodiments to immobilize the various components within body portion 55, as well as provide electrical insulation among the various components and between the components and a device operator. Cavity 56 can be partially filled, or not filled, as long as components within body portion 55 (such as cables 60, lead wires 80 and 81, as well as in certain embodiments ends of proximal portions 25 and 35) are immobilized relative to handle 50. Alternatively, sealing (including making air-tight an in certain embodiments liquid-proof) of openings 52 and 54 for prevention of fluid ingression into handle 50 can be sufficient to achieve the immobilization of components within body portion 55. Plug 58 can be fixedly coupled to body 55 to cap off cavity 56.

The handle designs of the present application allow cables as well as tubing to exit handle 50 distally (from the handle) at less than a 90 degree angle (including at an angle of zero degree or substantially parallel) to the longitudinal axis of handle 50 as well as probes 10. As such, the anchoring point for cables as well as tubing at handle 50 is much closer to distal end of device 100, thereby moving the center of gravity of device 100 closer to its distal end. In certain embodiments the described angles of tubing in relation to handle and described anchoring points can be applied to device 200 from FIG. 4 and to additional devices with three or more voltage delivery regions.

Such handle designs can minimize the possibility of cables as well as tubing entanglements. Such handle designs can also minimize adverse effects to probe placement as well as positioning caused by unintended forces. Such handle designs can further enable multiple devices to be positioned adjacent to each other so that the ablation volumes of the devices can merge into a combined ablation volume without voids.

Furthermore, devices incorporating distally exiting cables as well as tubing can reduce and can minimize the overall profile (including length as well as width) of the devices, as compared to other devices having proximally exiting cables. The reduced as well as minimized device profiles as disclosed herein can enable multiple devices with distally exiting cables as well as tubing to be used in a restricted operating space (including a biological imaging gantry). As such, the designs of the presently disclosed devices can enhance their applicability in treatment procedures involving, for example, biological imaging guidance (including CT-guidance). An advantage of a device with cables existing distally from the handle is that a center of gravity for the device is closer to the distal portion of the probe (closer to the tip than if the cables exit from the proximal portion of the handle); this adds stability and the probe will not be deflected as much (whether in use at a horizontal or vertical angle or between) during use.

Conventional handle designs have cables exiting a handle at its proximal end at an anile of 0 to 90 degrees relative to the longitudinal axis of the handle and the probe. While conventional handle designs do not provide the features of the above-described handle designs, they can be suitable for use with any of the probe designs described herein.

FIG. 13 provides a view of the distal portion of the device of FIG. 1 showing a blunt tip 84 for a bipolar device 100. Shown are the bipolar device 100 sections including the probe 10, the distal voltage delivery region 12, the electrically insulating region 13, the proximal voltage delivery region 14, the handle 50, the bundled cables 60, the connectors 66, and the blunt tip 84.

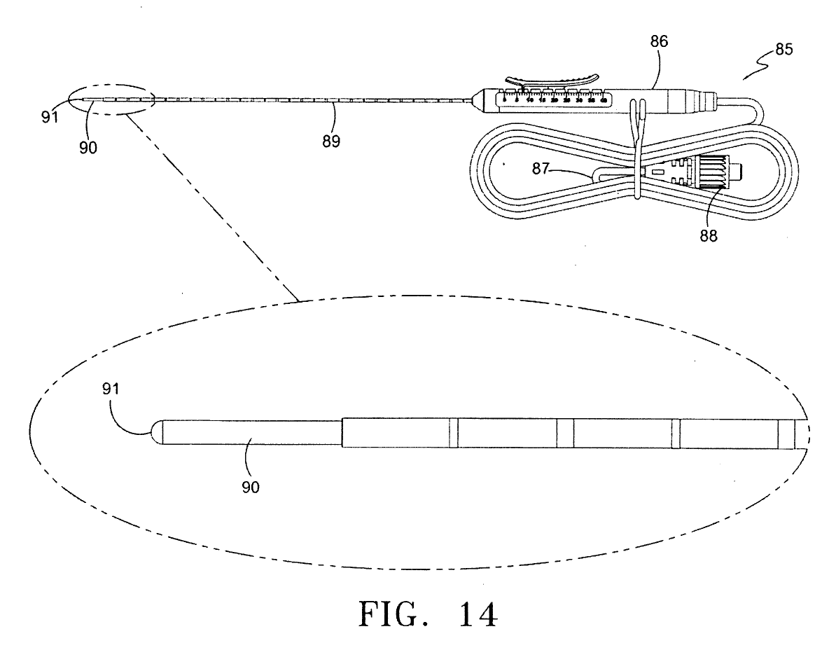

FIG. 14 provides a view of the distal portion of a monopolar device 85 having a blunt tip 91. Shown are the monopolar device 85 sections including the handle 86, the bundled cables 87, the connector 88, the probe 89, the voltage delivery region 90, and the blunt tip 91. The monopolar device involves ablation or other treatment of tissue through the application of energy from two probes each containing a single voltage delivery region here current can flow from one probe to the other probe for ablation and the probes can be independently placed to increase the effectiveness of treatment. In certain embodiments multiple probes are utilized together.

Specifically regarding FIGS. 13-14, the blunt tip shape can be of any known in the art necessary to adequately perform energy release, including but not limited to minimizing or preventing damage to surrounding cellular structures as well as increasing the effectiveness of energy release or the efficiency or the precision of treatment. In certain embodiments the blunt lip or the end of the tip for the device has a bullet-shape or a bullet-nosed shape. In other embodiments the blunt tip or the end of the tip can be rounded or have planes, be spherical or nonspherical in nature (including an end that appears as one-half of a sphere so that the end is smooth and curved), and can be ovoid in shape. The blunt tip can be utilized with multiple energy release forms. The blunt tip can be utilized as part of a device for electroporation, irreversible electroporation (each of nonthermal and thermal), as well as radiofrequency ablation, thermal electric heating, and traditional heating methods with electrodes using direct current or alternating current.

Also referring to FIGS. 13-14, the voltage delivery regions, the electrically insulating regions, and the voltage delivery members for the blunt tip embodiments of each of the bipolar and monopolar devices can be of equal diameters, substantially equal diameters, or unequal diameters. In certain embodiments the entire of the length of the probe for a device with a blunt tip is flush. In other embodiments the voltage delivery regions are substantially smaller or larger in diameter then the electrically insulating region.

Referring specifically to FIG. 14 cables 87 are shown as exiting the handle at the proximal end of the handle (closest to the user), though it is understood as described in this specification that the cables could exit at the distal end of the handle (adjacent to the proximal end of the probe) so as to provide stability and decrease the profile of the device, as is shown in FIG. 13 with the bipolar embodiment.

In various embodiments of FIGS. 13-14, voltage delivery regions can be independently polarized as well as independently energized so as to ensure that a circuit is formed for current movement from any of the voltage delivery regions to any of the voltage delivery regions. In certain embodiments a cathodic polarization in the voltage delivery region including the tip (at the distal end of the probe) will be energized such that current flows from the tip to a voltage delivery region independently charged anodically that is not at the tip and is closer to the proximal half of the probe (closes to the distal portion of the handle) than to the tip. In certain embodiments where multiple probes are utilized together (either monopolar, bipolar, or a combination of monopolar or bipolar with or without the blunt tip end), independently energizing and independently polarizing can be used to ensure current flows from any voltage delivery region on one probe to any voltage delivery region on another probe, at any point along the length of the voltage delivery member containing a voltage delivery region.

The blunt tip embodiment provides distinct advantages over the current state of the art. A blunt tip provides advantages of accuracy, precision, and exactness due to the non-cutting structure that will allow targeting of tissue without piercing of surrounding structures. A minimization of collateral damage occurs as nontarget tissue remains undamaged and minimally affected, providing the user increased degrees of freedom of action due to the non-piercing actions occurring with use of the blunt tip. In certain embodiments the blunt tip can be used to touch and even provide pressure to, change the shape of, or move a target structure or surrounding structure through the exertion of non-cutting and non-piercing pressure that will not pierce tissue or organ membranes and will not produce holes, tears, or punctures, thereby keeping the target structure intact.

In other embodiments the blunt tip will encounter nontarget tissue and will roll of the tissue, moving along the surface by partly revolving or turning. In various embodiments the blunt tip is utilized near nerve cells, parts of nerve cells, or nerve bundles, and can be used where nerve structural damage needs to be minimized or prevented. In various embodiments the blunt tip is used in regions where myelin sheath damage needs to be minimized or prevented. In additional embodiments the blunt tip can be used near or at biological structures of a conduit appearance, including but not limited to vessels or ureters to minimize or prevent damage. In yet other embodiments the blunt tip can be used at or near biological membranes to minimize or prevent damage.

The blunt tip can be utilized in certain embodiments in microsurgery, in areas where the tissues are delicate, thin, or otherwise prone to damage with current devices utilized in the art. In various embodiments the blunt tip is utilized in therapies, procedures, and surgeries of as well as surrounding the brain, where structures are delicate and where small openings are in many cases utilized for entry. In therapies surrounding the brain, the blunt tip provides the distinct advantage of providing leeway to users that have a buffer so as to prevent piercing of vital tissues. The blunt tip embodiments offer advantages in surgeries involving various brain structures, including but not limited to the parietal, occipital, temporal, and frontal lobes of the brain, the brainstem, the cerebellum, the ducts, glands, ventricles, as well as deep brain structures. In additional embodiments the blunt tip can be used, for example, in delicate surgeries involving dense vessel structures. Additional anatomical regions for therapies, procedures, and surgeries where the blunt tip can be utilized include physiologically homeostatic or pathological (including but not limited to tumors or cancers, benign or metastatic) portions of tissue of or surrounding adipose tissue, breast tissue, lymph (including lymph nodes and conduits), and ovarian tissue.

Yet other embodiments of blunt tip use include work involving tumor as well as cancer removal or treatment, either directly on cells as well as on surrounding cells and vasculature. Various embodiments of the blunt tip may also be utilized in therapies, procedures, and surgeries involving the abdomen as well as the bowels. These areas contain multiple regions of membranes and muscle groupings and are known as regions where ally piercings and tears can lead to medical disruptions requiring exploratory surgeries and which are difficult to find. The blunt tip would provide distinct advantages for work in these regions.

Various blunt tip embodiments also provide advantages to therapies, procedures, and surgeries of as well as around the lung, including the pleural space and pleural sac. Use of sharp instruments in this area is more prone to lead to a pneumothorax, and the result of accumulated gases and secondary responses and exposures can lead to additional infections and complications of lung collapse and other lung failures. The blunt tip embodiments remove this problem from work in these regions. The blunt tip can be utilized in other embodiments with or coupled to coatings or other devices increasing the ease of movement, entry, conductivity of energy, or otherwise increase the efficiency of the use of the device.

Additional advantages of the blunt tip embodiments surround the fact that released energy is not focalized. In certain embodiments the tip is part of the voltage delivery region or capable of conducting or releasing energy. In certain embodiments precision and accuracy and exactness are increased due to the fact the released energy is not concentrated into it very sharp tip which circumvents problems associated with nonequal energy release along the delivery region including the tip. The blunt tip provides for equal release of energy and prevents disruption of nontarget tissue as well as preventing unequal damage during therapies and within and around treatment areas. The blunt tip overcomes certain problems associated with the edge effect where high current densities occur in edges. This has been discussed in the following article, hereby incorporated by reference: Tungjitkusolmun S., Woo E., Cao H, Tsai J., Vorperian V., Webster J. “Finite Element Analysis of Uniform Current Density Electrodes for Radio-Frequency Cardiac Ablation.” IEEE Transactions on Biomedical Engineering, Vol. 47(1): 32-40 (2000). The blunt tip embodiments provide the advantage of decreasing arcing by minimizing aberrations of high current densities that can occur on material edges, and this decrease in arcing occurs regardless of whether the tip is charged anodically or even cathodically.

The size of the IRE device, with as well as without the blunt tip, can be that known in the art for treatment involving vessels and tissue of as well as within the prostate, uterus, lung, liver, kidney, brain, head, neck, bone, stomach, colon, pancreas, vascular, and duct. Treatment can include at least one of benign prostate hyperplasia (BHP), uterine fibroids, malignant masses. cancers, tumors, and benign tissues. Treatment can utilize at least one of percutaneous, laparoscopic, endoscopic, and natural orifice entry.

The blunt tip embodiment also provides advantages in treatments, therapies, and surgeries located in regions of the prostate, where delicate and precise ablations must be performed without damaging surrounding structures such as nerves that could lead to biological disruptions and dysfunctions. In certain embodiments the blunt tip device can be utilized in removal of or ablation of part or all of the prostate or tissues adjacent to or structurally supportive of the prostate.

In certain non-limiting examples, device 100 (or 200 from FIG. 4) as described herein can be used to ablate a predetermined volume of cells in a mammalian subject. Non-limiting treatment methods can involve two or more of the following non-limiting steps. Any one or more of the steps described herein can be taken place in any suitable as well as practical sequences as well as concurrently, without being limited thereto.

The methods of the present application can involve imaging (including but not limited to ultrasound, CT, MRI) of the target tissue volume to be ablated. The imaging can include but is not limited to one, two, or more two-dimensional or three-dimensional biological imaging modalities, such as ultrasonography (ultrasound), fluoroscopy, contrast-enhanced imaging, magnetic resonance imaging, tomographic imaging, ionizing radiation imaging, non-ionizing radiation imaging, gamma radiation imaging (using radioactive isotopes), positron emission tomography, projection radiography (including X-ray, using radiopaque contrast agent), photoacoustic imaging, tomography (including linear tomography, poly tomography, zonography, orthopantomography, computed tomography with or without enhancement using contrast agent), diffused optical imaging (using infrared wavelengths), elastography (using ultrasound, MRI, or CT), electrical impedance tomography, as well as optoacoustic imaging. The imaging can be carried out prior to, during, as well as after the tissue treatment using the devices. The imaging can provide constant feedback (including real-time feedback) for any portion of, or throughout, the image-guided treatment. The imaging can be used in part to identify the location of the target tissue volume, determine the desired ablation volume for selection of appropriate tissue treatment devices disclosed herein, as well as to identify appropriate point of entry (including puncture) for the probes thereof.

The methods of the present application can involve providing one or a combination of two or more tissue treatment devices disclosed herein that are suitable for ablating at least a portion of the desired ablation volume or its entirety. Criteria for selection of the appropriate tissue treatment devices include but are not limited to the configuration of the probe, voltage delivery parameters (such as predetermined voltage that in certain embodiments can be 2.5-3 kV), and predetermined electric field strength (including 0.25 kV/cm, 0.425 kV/cm, as well as 0.6 kV/cm).

The methods of the present application can involve coupling the tissue treatment device to a therapeutic energy source, such as a voltage pulse generator (including a high voltage pulse generator). Commercial providers for the therapeutic energy source include AngioDynamics Inc., (Queensbury, N.Y.). In one example, the tissue treatment device can be coupled to the therapeutic energy source such that voltage delivery region 12 can be cathodically energized and voltage delivery region 14 can be anodically energized to minimize arcing.

The methods of the present application can involve inserting a probe of the device at or about the predetermined point of entry into the subject. The insertion of the probe can be percutaneous, laparoscopic, endoscopic, as well as through natural orifices (including insertions related to orifice translumenal endoscopic surgery).

The methods of the present application can involve positioning any one, two, or more than two of the voltage delivery regions along the probe within or adjacent to the target tissue. The positioning can be carried out under image guidance using one or more of the biological imaging modalities disclosed herein. The design of the treatment devices disclosed herein maximizes stability of the positioned probe, minimizes inadvertent movements of the probe, enhances stability of the probe, and can minimize device failure due to kinks as well as breakage.

The methods of the present application can involve imaging the target tissue with the probe positioned for treatment or adjacent to the site to be treated. This imaging can be used to confirm the correct positioning of the probe, particularly the positioning of the one, two, or more than two voltage delivery regions. Repositioning and reimaging can be carried out until the desired positioning of the probe is achieved.

The methods of the present application can involve activating a therapeutic energy source. A therapeutic energy source can be configured to deliver therapeutic energy including high voltage pulses, and can be configured to optionally deliver testing energy, including low voltage pulses or high voltage pulses or with pulses that are each of a duration shorter than the duration of each therapeutic voltage pulse.

The methods of the present application can involve delivering a sufficient number of voltage ablation pulses from the therapeutic energy source through the probe to the target tissue. Each of the pulses can have the same or different duration, which can be on the order of 20 microseconds to 200 microseconds, and in certain embodiments can be from 30 microseconds to 100 microseconds. Each of the pulses can have the same or different voltage, which can be of various levels (including but not limited to 1 kV or greater, 2 kV or greater, 2.7 kV or greater, or 2.5 kV to 3 kV). Each of the pulses can have the same or different waveforms, such as square, triangle, sawtooth, sine, pulse, composite waveforms, and can be in the form of a Fourier series. Any two consecutive pulses can be separated by an inter-pulse duration of 0.15 seconds or greater (including but not limited to 0.2 seconds to 1 second or 0.25 seconds to 0.5 seconds). The pulses can be delivered together or separated into subsets of the same or different number of pulses (including but not limited to subsets of 1 to 10 pulses each). The pulses can be the same or different in pulse duration, waveform, as well as voltage as well as amplitude within each subset as well as between different subsets. The ablation treatment that is sufficient to ablate the predetermined ablation volume can require just one pulse or a few pulses if the ablation volume is small, and 10 or more pulses for medium to large ablation volumes (in certain embodiments being 20 or more, 50 or more, 90 or more, 100 or more, 150 or more, 500 or less, 300 or less, or optionally 200 or less). A “train” is a term used to state a series of sequential electrical pulses. The ablation treatment that is sufficient to ablate the predetermined ablation volume can be delivered within 6 minutes (in certain embodiments being 5 minutes or less, or 2 minutes or less, or 1 minute or less). A non-limiting voltage ablation regimen can include multiple trains (in one embodiment 9 trains) of multiple pulses (in one embodiment 10 pulses) each, with pulse duration being 100 μs or shorter, pulse waveform being square, duration between consecutive pulses being 0.25 seconds, and duration between consecutive trains being 3 seconds. Control of parameters such as pulse durations, inter-pulse durations, voltage, amplitude, pulse waveform, and number of pulses can be handled by the therapeutic energy source. Such control can be carried out automatically according to preset values, based on the sensor (including information from the sensor including but not limited to impedance, current, voltage, chemical concentrations, pH, as well as ionic strength) feedbacks, as well as through manual input by the device operator. In certain embodiments, the application of the pulsed electric fields includes pulses of 1.5 kV/cm in 3 trains of 10 pulses each to ablate tissue.

The methods of the present application can involve exposing cells within the predetermined ablation volume to pulsed high intensity electric fields delivered from the probe. The electric fields can cause irreversible electroporation and subsequent cell death of the predetermined ablation volume. The effects of the ablation can be substantially immediately detectable (as well as detectable almost immediately referring to a very slight delay) following the pulsed voltage ablation delivery.

The methods of the present application can involve terminating the delivery of voltage ablation pulses based on predetermined criteria as well as based on feedback signals. Feedback signals can include sensor feedback signals, visual confirmation of predetermined desirable changes by the operator, or combinations thereof.

The methods of the present application can involve repeating the steps of probe positioning and therapeutic voltage pulse delivery to treat other selected treatment volumes. The other treatment volumes can overlap, partially overlap, or alternatively not overlap with the prior ablation volume.

The methods of the present application can involve retracting the probes partially or fully from the subject once all selected treatment volumes are treated. The probe track can be cauterized as understood by one of ordinary skill in the art, if desired.

Operating parameters suitable for use with the voltage delivery devices of the present application to ablate selected volumes of tissues (such as those depicted in FIGS. 5-10, but not limited thereto) using pulsed electric fields include but are not limited to: amplitude of voltage pulses, duration of each pulse, total number of voltage pulses, and duration between consecutive pulses. Amplitude of voltage pulses can be 1 kV or higher (in certain embodiments being at least one of: 2 kV or higher, 2.5 kV or higher, 2.7 kV or higher, 3 kV or higher, 5 kV or higher).

Duration of each pulse can be 100 microseconds or shorter (including but not limited to 50 microseconds or shorter or alternatively 20 microseconds or shorter). Certain embodiments include short pulses that would be more than sufficient to achieve any clinically relevant ablation, such as a total voltage exposure duration of one second (such as 10,000 pulses of 100 microseconds each).

It is noted that the tissue in the ablation volume subjected to pulsed electric field ablation of the present application is in its entirety exposed to ablative electric fields at the same time, albeit at different field strengths. Utilizing the electric field application effectively can lead to shortened procedures that can be 6 minutes or shorter.

In one embodiment, 30 trains of 10 pulses each can be used, with a pulse duration of 100 microseconds, a duration between consecutive pulses of 1 second, and a duration between consecutive trains of 3 seconds. In another embodiment, 300 pulses can be used, with a pulse duration of 100 microseconds and a duration between consecutive pulses of 1 second. In a further embodiment, 9 trains of 10 pulses each can be used, with a pulse duration of 100 microseconds, a duration between consecutive pulses of 0.25 seconds, and a duration between consecutive trains of 3 seconds.

The disclosed pulsed electric field ablation, when carried out under certain parameters and operating conditions, can selectively spare (including without damaging, destroying or denaturing) certain tissues and structures present within the ablation volume. Non-limiting tissues that can be selectably spared by the pulsed electric field ablation include nervous, vascular, duct, as well as collagen-rich tissues.

The total number of voltage pulses necessary to ablate a particular cell within a target tissue can depend on aspects such as cell shape and size within the target tissue, strength of the electric field the cell is subjected to, and duration of each pulse. In a qualitative but not necessarily quantitative example for illustration only, a cell requiring a certain number of pulses (such as 100) each of a certain duration (such as 100 microseconds) at a certain field strength (such as 0.4 kV/cm) to be successfully be ablated can also be ablated when subjected to a smaller number of pulses (such as 60-70) each of substantially the same or shorter duration (such as 20-100 microseconds) at a higher field strength (such as 0.6 kV/cm), or to substantially the same number of pulses (such as 100) each of a shorter duration (such as 20-50 microseconds) at substantially the same field strength (such as 0.4 kV/cm).

In another qualitative but not necessarily quantitative example for illustration only, probe 10 of FIG. 1 with a configuration leading to the FEA from FIG. 5 can generate an ablation volume having a diameter of 2 cm by a length of 3.5 cm by delivering 90 pulses of 100 microseconds each at 2.7 kV as a treatment. For a first cell located at the border of this ablation volume (such as 1 cm away from probe 10) subjected to a first electric field strength of 0.25 kV/cm during the treatment, all 90 of the pulses can be necessary for ablation of the first cell. However a second cell within this ablation volume (such as located 0.5 cm away from probe 10) subjected to a second electric field strength of 1 kV/cm during the treatment, can be ablated with a fewer number of pulses (such as only 40 pulses).

Duration between consecutive pulses can be equal to or longer than duration of muscle contractions (typically 50-160 milliseconds) to allow muscle cells to substantially recover following each voltage pulse, and to allow substantial dissipation of thermal buildup, it any, as a result of the voltage pulse. Duration between consecutive pulses can be substantially longer than duration of each pulse, such as 2,000-fold or greater. Duration between consecutive pulses can be 0.15 seconds or longer, or in alternative embodiments can be 0.2 seconds or longer (equivalent to a pulse frequency of lower than 5 Hz), or 0.25 seconds or longer (equivalent to a pulse frequency of 4 Hz or lower), or 1 second or longer.

Unless otherwise defined herein, scientific and technical terminologies employed in the present disclosure shall have the meanings that arc commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims. the terms “at least one” and “one or more” have the same meaning and include one, two, three or more.

Other than in the operating examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth can be varied.

Notwithstanding that the numerical ranges and parameters setting forth the broad scope of the disclosure are approximations, the numerical values set forth in the specific examples are reported as precisely as possible. Any numerical value, however, inherently contains limitations as to testing measurements. Furthermore, when numerical ranges of varying scope are set forth herein, it is contemplated that any combination of these values inclusive of the recited values can be used.

Examples provided herein, including those following “such as” are considered as illustrative only of various aspects and features of the present disclosure and embodiments thereof, without limiting the scope of any of the referenced terms or phrases either within the context or outside the context of such descriptions. Any suitable equivalents, alternatives, and modifications thereof (including materials, substances, constructions, compositions, formulations, methods, as well as conditions) known as well as available to one skilled in the art can be used or carried out in place of or in combination with those disclosed herein, and are considered to fall within the scope of the present disclosure. All these alternatives and variations are intended to be included within the scope of the claims where the terms “comprising”, “formed from” and “formed of” all denote open claim language. Those familiar with the art can recognize other equivalents to the specific embodiments described herein which equivalents are also intended to be encompassed by the claims.

Throughout the present disclosure in its entirety, any and all of the one, two, or two or more features and aspects disclosed herein, explicitly or implicitly, following terms “example”, “examples”, “such as,” and the likes thereof can be practiced in any combinations of two, three, or more thereof (including their equivalents, alternatives, and modifications), whenever and wherever appropriate as understood by one of ordinary skill in the art. Some of these examples are themselves sufficient for practice singly (including their equivalents, alternatives, and modifications) without being combined with any other features, as understood by one of ordinary skill in the art. Therefore, specific details disclosed herein are not to be interpreted as limiting, but merely as a basis for the claims and as a representative basis for teaching one skilled in the art to variously employ aspects and features of the present disclosure in any appropriate manner.

1. A tissue treatment device comprising:
a means for treating tissue using non-thermal irreversible electroporation, wherein the means comprises a probe having
a proximal end and a distal end;
at least a first voltage delivery member disposed along the entire length of the probe, wherein the first voltage delivery member has first voltage delivery region capable of delivering a voltage for irreversible electroporation;
at least a second voltage delivery member disposed along the probe and positioned coaxially in relation to the first voltage delivery member, wherein the second voltage delivery member has a second voltage delivery region capable of delivering a voltage for irreversible electroporation; and
at least one electrically insulating region coaxially disposed in relation to the first voltage delivery member.

2. The device of claim 1, Wherein the at least one electrically insulating region is positioned between at least a portion of the first voltage delivery region and at least a portion of the second voltage delivery region.

3. The device of claim 2, wherein the first voltage delivery region is substantially the same length as the second voltage delivery region.

4. The device of claim 2, wherein the first voltage delivery region is of a substantially different length than the second voltage delivery region.

5. The device of claim 2, wherein at least one of the first delivery region and the second voltage delivery region is substantially the same length as the at least one electrically insulating region.

6. The device of claim 2, wherein at least one of the first delivery region and the second voltage delivery region is of a substantially different length than the electrically insulating region.

7. The device of claim 1, wherein the device comprises a plurality of voltage delivery regions.

8. The device of claim 1, wherein the device comprises a plurality of electrically insulating regions.

9. The device of claim 8, wherein the plurality of electrically insulating regions comprises a first electrically insulating region and a second electrically insulating region.

10. The device of claim 9, wherein the first electrically insulating region has substantially the same length as the second electrically insulating region.

11. The device of claim 9, wherein the first electrically insulating region has a substantially different length than the second electrically insulating region.

12. The device of claim 1, wherein at least one of the first voltage delivery region and the second voltage delivery region comprises a single tip, wherein the tip is positioned at the distal end of the probe, such that the tip is capable of piercing tissue.

13. The device of claim 12, wherein the tip is a sharpened tip.

14. The device of claim 1, wherein at least one of the first voltage delivery region and the second voltage delivery region comprises a single tip, wherein the tip is positioned at the distal end of the probe, such that during treatment the tip is capable of contacting non-target tissue while allowing the non-target tissue to remain intact.

15. The device of claim 14, wherein the single tip is a blunt tip.

16. The device of claim 2, wherein the first voltage delivery region and the second voltage delivery region have an outer diameter, and the at least one electrically insulating region has an outer diameter, and wherein the outer diameter of the first and second voltage delivery regions is substantially equivalent to the outer diameter of the electrically insulating region.

17. The device of claim 2, wherein at least one of the first voltage delivery member and the second voltage delivery member comprises at least one lumen.

18. The device of claim 17, wherein the at least one lumen is capable of delivering substances to tissue, Wherein the substances are selected from the group consisting of fluids, media, solutions, suspensions, therapeutics, hydrogels, nanoparticles, and microparticles.

19. The device of claim 1, wherein the device is capable of extracting a substance selected from the group consisting of tissue, fluids, medium, solutions, suspensions, therapeutics, hydrogels, nanoparticles, and microparticles.

20. The device of claim 2, wherein at least one of the first voltage delivery region and the second voltage delivery region is independently energized.

21. The device of claim 2, wherein at least one of the first voltage delivery region and the second voltage delivery region is independently polarized.

22. The device of claim 2, wherein at least one of the first voltage delivery region and the second voltage delivery region is capable of applying at least one pulsed electric field gradient to target tissue at a level that is sufficient to non-thermally irreversibly electroporate the target tissue.

23. The device of claim 22, wherein the target tissue is selected from the group consisting of prostate, uterus, lung, liver, kidney, brain, head, neck, bone, stomach, colon, pancreas, vascular, adipose, lymph, breast, ovarian, duct, and mammalian.

24. The device of claim 22, wherein the target tissue comprises a mass of tissue selected from the group consisting of benign prostate hyperplasia (BPH), uterine fibroids, malignant tissues, cancers, tumors, and benign tissues.

25. The device of claim 1, wherein the device further comprises
a handle having
a proximal end and a distal end;
a handle body; and
at least one cable, wherein the at least one cable is positioned within at least a portion of the handle body, such that the at least one cable is adjacent to the proximal end of the probe, and at least a portion of the at least one cable extends outwardly from the distal end of the handle toward the distal end of the probe.

26. A tissue treatment device comprising:
a means for treating tissue using non-thermal irreversible electroporation, wherein the means comprises a probe having
a proximal end and a distal end;
a blunt tip disposed at the distal end of the probe;
a handle disposed at the proximal end of the probe;
a first voltage delivery member disposed along the entire length of the probe, wherein the first voltage delivery member has a first voltage delivery region capable of delivering a voltage for irreversible electroporation; and
a second voltage delivery member disposed along the probe and positioned coaxially in relation to the first voltage delivery member, wherein the second voltage delivery member has a second voltage delivery region capable of delivering a voltage for irreversible electroporation; and
at least one electrically insulating region coaxially disposed in relation to the first voltage delivery member.

27. A tissue treatment device comprising:
a probe having
a proximal end and a distal end;
a first voltage delivery member disposed along the entire length of the probe, wherein the first voltage delivery member has a first voltage delivery region; and
a second voltage delivery member disposed along the probe and positioned coaxially in relation to the first voltage delivery member, wherein the second voltage delivery member has a second voltage delivery region; and
at least one electrically insulating region coaxially disposed in relation to the first voltage delivery member;
a blunt tip disposed at the distal end of the probe; and
a handle disposed at the proximal end of the probe, wherein the handle has a proximal end and a distal end and a handle body.

28. The device of claim 27, wherein the device further comprises at least one cable, wherein at least a portion of the at least one cable is positioned within at least a portion of the handle body, such that the at least one cable is adjacent to the proximal end of the probe, and at least a portion of the at least one cable extends outwardly from the distal end of the handle toward the distal end of the probe.

29. The device of claim 27, wherein the cables are positioned such that a profile of the device is maintained, and a center of gravity of the device is present distally of the distal end of the handle.

30. The device of claim 28, wherein the cables are positioned such that a profile of the device is maintained, and a center of gravity of the device is present distally of the distal end of the handle.

31. A tissue treatment device comprising:
a first probe having
a proximal end and a distal end;
a first voltage delivery member disposed along the entire length of the probe, wherein the first voltage delivery member has a first voltage delivery region; and
a first blunt tip disposed at the distal end of the probe; and
a first handle disposed at the proximal end of the probe, wherein the first handle has a proximal end and a distal end and a first handle body.
a second probe having
a second voltage delivery member disposed along the entire length of the probe, wherein the second voltage delivery member has a second voltage delivery region; and
a second blunt tip disposed at the distal end of the probe; and
a second handle disposed at the proximal end of the probe, wherein the handle has a proximal end and a distal end and a second handle body.

32. A method of tissue treatment using irreversible electroporation, comprising:
providing a tissue treatment device wherein the device comprises:
a proximal end a distal end;
a first voltage delivery member disposed along the entire length of the probe, wherein the first voltage delivery member has a first voltage delivery region capable of delivering a voltage for irreversible electroporation of a selected volume of tissue; and
a second voltage delivered member disposed along the probe and positioned coaxially in relation to the first voltage delivery member, wherein the second voltage delivery member has a second voltage delivery region capable of delivering a voltage for irreversible electroporation; and
at least one electrically insulating region coaxially disposed in relation to the first voltage delivery member;
positioning the device within the selected volume of tissue;
delivering pulsed electric field gradients to the selected volume of tissue; and
ablating the selected volume of tissue using non-thermal irreversible electroporation.

33. The method of claim 31, wherein the device is positioned using a technique selected from the group consisting of percutaneous, laparoscopic, endoscopic, and natural orifice entry.

34. The method of claim 31, wherein the step of delivering comprises applying at least one pulsed electric field gradient to the target tissue at a level that is sufficient to non-thermally irreversibly electroporate the target tissue.

35. The method of claim 31, wherein the target tissue is selected from the group consisting of prostate, uterus, lung, liver, kidney, brain, head, neck, bone, stomach, colon, pancreas, vascular, adipose, lymph, breast, ovarian, duct, and mammalian.

36. The method of claim 31, wherein the target tissue comprises a mass of tissue selected from the group consisting of benign prostate hyperplasia (BPH), uterine fibroids, malignant tissues, cancers, tumors, and benign tissues.

37. The method of claim 33, wherein the pulsed electric field gradients are within the range of approximately 0.075 kV/cm to approximately 3 kV/cm.

38. The method of claim 33, wherein the pulsed electric field gradients are applied such that a duration of between approximately 0.15 seconds and 0.5 seconds exists between each pulsed electric field gradient.

39. The method of claim 33, wherein the temperature of the selected volume of tissue remains below approximately 50° C. during treatment.

40. The method of claim 33, wherein duration of each pulsed electrical field gradient is between approximately 20 microseconds and 200 microseconds.

41. The method of claim 39, wherein the duration of each pulsed electrical field gradient is approximately 100 microseconds.

42. The method of claim 33, wherein the pulsed electrical field gradients are delivered in a train consisting of a series of approximately ten sequential pulses, with a duration of between approximately 0.25 and 3 seconds between each pulse.

43. The method of claim 41, wherein between approximately six and thirty trains are applied to ablate the tissue.

44. A method of tissue treatment, comprising:
providing a tissue treatment device wherein the device comprises:
a probe having
a proximal end and a distal end;
a first voltage delivery member disposed along the entire length of the probe, wherein the first voltage delivery member has a first voltage delivery region; and
a second voltage delivery member disposed along the probe and positioned coaxially in relation to the first voltage delivery member, wherein the second voltage deliver member has a second voltage delivery region; and
at least one electrically insulating region coaxially disposed in relation to the first voltage delivery member probe,
a blunt tip disposed at the distal end of the probe; and
a handle disposed at the proximal end of the probe, wherein the handle has a proximal end and a distal end and a handle body;
positioning the device within the selected volume of tissue;
delivering pulsed electric field gradients to the selected volume of tissue; and
ablating the selected volume of tissue using non-thermal irreversible electroporation.

45. The method of claim 43, wherein the target tissue is selected from the group consisting of prostate, uterus, lung, liver, kidney, brain, head, neck, bone, stomach, colon, pancreas, vascular, adipose, lymph, breast, ovarian, duct, and mammalian.

46. The method of claim 43, wherein the target tissue comprises a mass of tissue selected from the group consisting of benign prostate hyperplasia (BPH), uterine fibroids, malignant tissues, cancers, tumors, and benign tissues.
